(19) United States
(12) Patent Application Publication
(10) Pub. No.: US 2023/0154600 A1
(43) Pub. Date: May 18, 2023

(54) MONITORING ASSISTANCE DEVICE, MONITORING ASSISTANCE SYSTEM, MONITORING ASSISTANCE METHOD, AND STORAGE MEDIUM

(71) Applicant: CANON MEDICAL SYSTEMS CORPORATION, Otawara-shi (JP)

(72) Inventor: Shinya SUGIYAMA, Nasushiobara (JP)

(73) Assignee: CANON MEDICAL SYSTEMS CORPORATION, Otawara-shi (JP)

(21) Appl. No.: 18/054,732

(22) Filed: Nov. 11, 2022

(30) Foreign Application Priority Data
Nov. 17, 2021 (JP) 2021-187303

(51) Int. Cl. G16H 40/20; A61B 5/00

(52) U.S. Cl. CPC G16H 40/20; A61B 5/742; A61B 5/0205

(57) ABSTRACT

A monitoring assistance device of an embodiment includes processing circuitry. The processing circuitry is configured to acquire an execution status of at least one of an operation related to use of an apparatus that acquires biometric information of a managed person or an operation other than the operation related to use, determine, on the basis of the execution status of the managed person and a workflow defining an operation timing of the apparatus, whether or not the operation has been executed at the operation timing, and control a notification device that outputs a notification including information on the operation of the apparatus on the basis of whether or not the operation has been executed.

### CROSS-REFERENCE TO RELATED APPLICATION

The present application claims priority based on Japanese Patent Application No. 2021-187303 filed Nov. 17, 2021, the content of which is incorporated herein by reference.

### FIELD

Embodiments disclosed in the present description and drawings relate to a monitoring assistance device, a monitoring assistance system, a monitoring assistance method, and a storage medium.

### BACKGROUND

In recent years, progress has been made in monitoring biometric information of patients (hereafter referred to as “managed persons”) who stay in places such as their homes away from medical facilities and are managed by medical institutions. In monitoring biometric information of a managed person, for example, the managed person himself/herself acquires the biometric information through measurement using a measurement device or the like. The acquired biometric information is automatically transmitted to, for example, a medical facility.

Although biometric information is acquired by a managed person himself/herself through measurement in biometric information monitoring, the managed person is not necessarily skilled at carrying out biometric information monitoring. For this reason, it is sometimes difficult for the managed person to know when and how to perform operations such as measurement using a measurement device and preparation of the measurement device.

### BRIEF DESCRIPTION OF THE DRAWINGS

FIG. 1 is a configuration diagram of a physical condition management system 1 according to an embodiment.

FIG. 2 is a perspective view showing a guide sheet 31 on which a measurement device 10 is placed.

FIG. 3 is a plan view showing the guide sheet 31 on which the measurement device 10 is placed.

FIG. 4 is a diagram showing an example of a device workflow.

FIG. 5 is a flowchart showing an example of processing of a monitoring assistance device 100.

FIG. 6 is a diagram showing display content of a monitor 40.

FIG. 7 is a diagram showing display content of the monitor 40.

FIG. 8 is a diagram showing display content of the monitor 40.

FIG. 9 is a diagram showing display content of the monitor 40.

FIG. 10 is a diagram showing display content of the monitor 40.

### DETAILED DESCRIPTION

Hereinafter, a monitoring assistance device, a monitoring assistance system, a monitoring assistance method, and a storage medium according to embodiments will be described with reference to the drawings.

A monitoring assistance device of an embodiment includes processing circuitry. The processing circuitry is configured to acquire an execution status of at least one of an operation related to use of an apparatus that acquires biometric information of a managed person or an operation other than the operation related to use, determine, on the basis of the execution status of the managed person and a workflow defining an operation timing of the apparatus, whether or not the operation has been executed at the operation timing, and control a notification device that outputs a notification including information on the operation of the apparatus on the basis of whether or not the operation has been executed. According to the monitoring assistance device of the embodiment, it is possible to prompt the managed person to perform an operation of a measurement device.

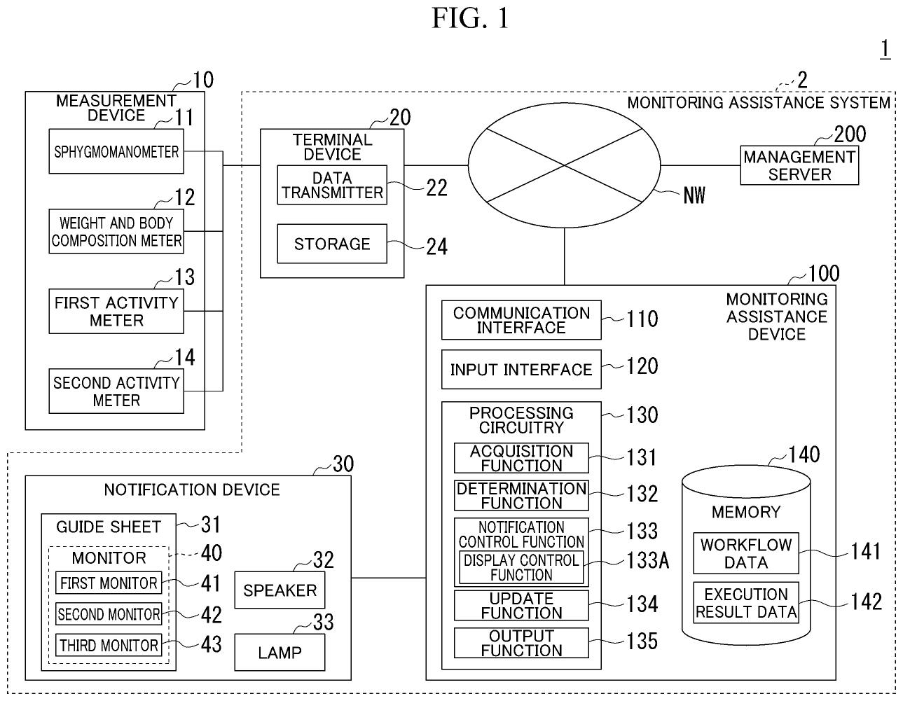

FIG. 1 is a configuration diagram of a physical condition management system 1 of an embodiment. The physical condition management system 1 includes, for example, a monitoring assistance system 2 and a measurement device 10. The monitoring assistance system 2 includes, for example, a terminal device 20, a notification device 30, a monitoring assistance device 100, and a management server 200. The monitoring assistance system 2 is, for example, a system for measuring biometric information of a managed person using the measurement device 10 and allowing a doctor or the like to manage the physical condition of the managed person using the measured biometric information.

The measurement device 10, the terminal device 20, the monitoring assistance device 100, and the management server 200 can transmit/receive data to/from each other via a communication network NW using, for example, a wireless LAN, a cellular network, or a Wi-Fi network. The communication network NW is a general information communication network using telecommunication technology. The communication network NW includes a telephone communication line network, an optical fiber communication network, a cable communication network, a satellite communication network, and the like in addition to a wireless/wired LAN such as a hospital backbone local area network (LAN) and the Internet. The measurement device 10 is an example of an apparatus that acquires biometric information.

The physical condition management system 1 includes a plurality of types of measurement devices 10. The physical condition management system 1 includes, as measurement devices 10, a sphygmomanometer 11, a weight and body composition meter 12, a first activity meter 13, and a second activity meter 14, for example. The sphygmomanometer 11 measures the blood pressure of the managed person as biometric information using, for example, a belt wrapped around an arm of the managed person. The managed person is an example of a living body, and the sphygmomanometer 11 is an example of a measurement apparatus. Blood pressure as a measurement result measured by the sphygmomanometer 11 is an example of biometric information.

The weight and body composition meter 12 functions, for example, when the managed person is on the weight and body composition meter 12, and measures the weight and body composition of the managed person as biometric information. The first activity meter 13 is installed and used, for example, in the home or the like of the managed person. The first activity meter 13 measures the amount of activity of the managed person as biometric information.

The first activity meter 13 is, for example, carried around by the managed person during the daytime and released from the managed person at night. Both the weight and body composition meter 12 and the first activity meter 13 are examples of a measurement apparatus. Weight and body composition as measurement results measured by the weight and body composition meter 12 and the amount of activity as a measurement result measured by the first activity meter 13 are examples of biometric information.

The second activity meter 14 is used by being worn, for example, on a part of the managed person, for example, a wrist. The second activity meter 14 measures the amount of activity of the managed person as biometric information. The second activity meter 14 has a charging function. The second activity meter 14 is worn by the managed person at all times, for example, except during charging.

At the time of charging, the second activity meter 14 is removed from the managed person and charged by being connected to a charging device. The second activity meter 14 may be charged while being worn by the managed person. The second activity meter 14 is an example of a measurement device. The second activity meter 14 is an example of a chargeable apparatus. The amount of activity as a measurement result measured by the second activity meter 14 is an example of biometric information.

The measurement device 10 outputs measurement information indicating a measurement result of each piece of biometric information to the terminal device 20. The measurement information of the measurement device 10 can be output to the terminal device 20 through communication via the communication network NW. The measurement information of the measurement device 10 may be output to the terminal device 20 via short-range wireless communication such as wired, near field communication (NFC), Bluetooth (registered trademark), dedicated short range communication (DSRC), infrared communication, or the like. The measurement information of the measurement device 10 may be stored in a universal serial bus (USB) and output to the terminal device 20 via the USB.

The terminal device 20 starts an application program, a browser, or the like for using services provided by the physical condition management system 1 and supports the services provided by the physical condition management system 1. In the following description, it is assumed that the terminal device 20 is a mobile terminal such as a tablet or smartphone, and an application program (service application) for receiving services is started. The terminal device 20 may be a terminal other than a mobile terminal The terminal device 20 may be, for example, a personal computer.

The terminal device 20 includes, for example, a data transmitter 22 and a storage 24. The data transmitter 22 transmits measurement information output by the measurement device 10 to the management server 200. When the measurement information is output, the data transmitter 22 transmits, to the monitoring assistance device 100, operation completion information indicating that an operation has been executed as an execution status of at least one of an operation related to use of the measurement device 10 (hereinafter referred to as a use-related operation) and an operation other than the use-related operation (hereinafter referred to as a non-use-related operation).

The operation of the measurement device 10 differs depending on the measurement device 10. For example, a use-related operation of the sphygmomanometer 11 is measurement of blood pressure. A use-related operation of the weight and body composition meter 12 is measurement of a weight and body composition. A use-related operation of the first activity meter 13 is attachment of the first activity meter 13 to the managed person or removal of the first activity meter 13 from the managed person.

A non-use-related operation of the second activity meter 14 is starting charging and ending charging. Starting charging is, for example, connecting the second activity meter 14 to a charging device, and ending charging is, for example, removing the second activity meter 14 from the charging device. The storage 24 stores and saves measurement information output by the measurement device 10.

The use-related operation of the measurement device 10 is an operation related to the use of the measurement device 10 to exhibit the function thereof and, for example, the use-related operation of the sphygmomanometer 11 is an operation of measuring blood pressure. Therefore, for example, an operation of using a function displayed on the sphygmomanometer 11 to use the sphygmomanometer 11 as a clock to measure time does not correspond to the use-related operation of the sphygmomanometer 11. Taking the second activity meter 14 as an example, the use-related operation of the second activity meter 14 is an operation of measuring the amount of activity of a user, and thus starting and ending charging of the second activity meter 14 is a non-use-related operation of the second activity meter 14.

Further, an execution status of an operation of the measurement device 10 differs depending on the measurement device 10. For example, an execution status of a use-related operation of the sphygmomanometer 11 is a measurement status of measuring the blood pressure of a subject with the sphygmomanometer 11, and an execution status of a use-related operation of the weight and body composition meter 12 is a measurement status of measuring the weight and body composition of a subject with the weight and body composition meter 12. An execution status of a use-related operation of the first activity meter 13 is a status of the first activity meter 13 worn on a subject and a status of the first activity meter 13 removed from the subject, and an execution status of a non-use-related operation of the second activity meter 14 is a charging start status and a charging end status for the second activity meter 14.

Instead of or in addition to the aspect in which the terminal device 20 transmits the operation completion information, the monitoring assistance device 100 may receive information such as measurement information and the like transmitted from the measurement device 10 to the terminal device 20 as operation completion information. The operation completion information is, for example, information included in data communication between the terminal device 20 and the management server 200. The operation completion information includes, for example, information on the time when the operation of the measurement device 10 has been performed, information on an identifier (ID) attached to the measurement device 10 as identification information, and the like, in addition to measurement information.

The notification device 30 and the monitoring assistance device 100 are connected by wire, for example. The notification device 30 may receive control information transmitted by the monitoring assistance device 100 through communication via the communication network NW using a wireless LAN, a cellular network, or a Wi-Fi network. The control information transmitted by the monitoring assistance device 100 may be transmitted to the notification device 30 via short-range wireless communication such as NFC, Bluetooth (registered trademark), DSRC, or infrared communication.

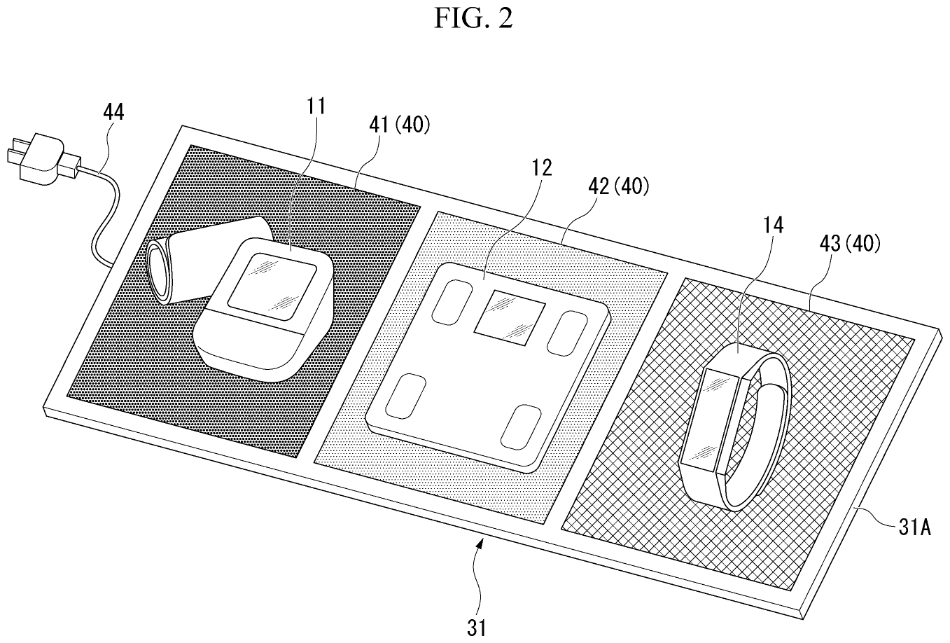

The notification device 30 includes, for example, a guide sheet 31, a speaker 32, and a lamp 33. FIG. 2 is a perspective view showing the guide sheet 31 on which the measurement devices 10 are placed. The guide sheet 31 includes a sheet body 31A. Three monitors, that is, a first monitor 41, a second monitor 42, and a third monitor 43, are provided as a monitor 40 on the sheet body 31A.

The first monitor 41, the second monitor 42, and the third monitor 43 are arranged side by side in close proximity (adjacent) to each other. The measurement devices 10 are placed on the guide sheet 31. The guide sheet 31 is a dedicated sheet for placing the measurement devices 10 and displaying information related to operations of the measurement devices 10 (hereinafter referred to as operation-related information). The guide sheet 31 is an example of a placing part.

A specific measurement device 10 is associated with each of the first monitor 41 to the third monitor 43. The sphygmomanometer 11 is associated with the first monitor 41. The weight and body composition meter 12 is associated with the second monitor 42. The second activity meter 14 is associated with the third monitor 43.

A first device such as the sphygmomanometer 11 is placed on the first monitor 41. A second device such as the weight and body composition meter 12 is placed on the second monitor 42. A third device such as the second activity meter 14 is placed on the third monitor 43. A power cord 44 extends from the sheet body 31A. Although the monitor 40 is not associated with the first activity meter 13, notification with respect to the first activity meter 13 is performed through the speaker 32 or the lamp 33, for example. The monitor 40 is an example of a display device. The first monitor 41 to the third monitor 43 are examples of an information display.

The speaker 32 and the lamp 33 are provided in the terminal device 20, for example. Although the speaker 32 and the lamp 33 are not provided on the guide sheet 31, the speaker 32 and the lamp 33 may be provided on the guide sheet 31. The speaker 32 and the lamp 33 may be provided independently of the guide sheet 31 and the terminal device 20. Display of the first monitor 41 to the third monitor 43, output of sound and electronic sound from the speaker 32, and lighting and blinking of the lamp 33 are controlled by the monitoring assistance device 100.

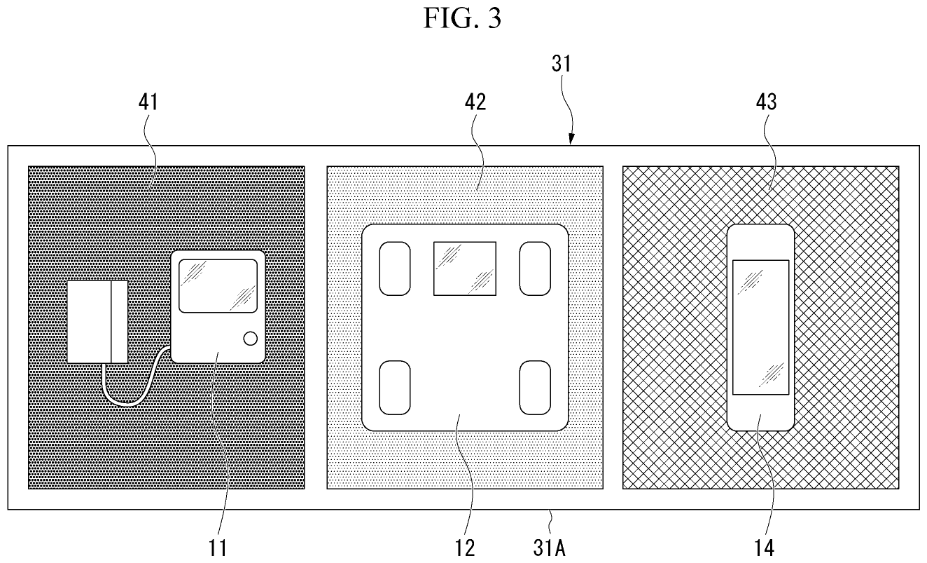

FIG. 3 is a plan view showing the guide sheet 31 on which the measurement devices 10 are placed. Operation-related information of the respective measurement devices 10, in this case, colors are displayed on the first monitor 41 to the third monitor 43. A notification control function 133 causes the monitor 40 to display different information, for example, different colors, depending on execution statuses of operations of the measurement devices 10 as notification of the operation-related information. The operation-related information may be other than color.

The operation-related information may be represented by, for example, lighting and blinking modes and a blinking speed difference, or may be represented by messages. In addition, the operation-related information may be represented by different sounds among the sounds output by the speaker 32, may be represented by lighting and blinking modes and a blinking speed difference in the lamps 33, lighting and blinking points when a plurality of lamps 33 are provided, and the like.

In the example shown in FIG. 3, the first monitor 41 is turned off in order to indicate a completed (measured, charged) state. The completed state may be displayed in a particular first color, for example, green. The completed state in this case may be displayed, for example, by lighting. The second monitor 42 is displayed in a second color, for example, orange, to indicate an execution timing (measurement timing, charging timing) state. The execution timing state is displayed, for example, by blinking.

The third monitor 43 is displayed in a third color, such as pink, which indicates an execution standby (measurement standby, charging standby) state. The execution standby state is displayed, for example, by lighting. The execution standby state is, for example, notification when an operation execution time has not come yet. Although a monitor corresponding to the first activity meter 13 is not provided, a fourth monitor corresponding to the first activity meter 13 may be provided on the guide sheet 31.

In addition, the third monitor 43 may display a fourth color, such as light blue, which indicates an in-progress (charging) state. Further, the monitor 40 displays an execution promotion state when the managed person forgets to operate the measurement device 10 after the execution timing state. The execution promotion state is, for example, display of blinking the display of the color displayed in the execution timing state. Display of the execution promotion state is an example of reminder information. Lighting and blinking modes of respective notifications may be different modes. Lighting and blinking in the completed state, the execution timing state, the execution standby state, the in-progress state, and the execution promotion state may be changed (replaced), or the blinking speed at the time of blinking the monitor 40 may be changed.

The monitoring assistance device 100 is included in, for example, a personal computer of the managed person. The monitoring assistance device 100 is installed in the home of the managed person, for example. The monitoring assistance device 100 supports services provided by the physical condition management system 1 by starting, for example, an application program, a browser, or the like for using the services provided by the physical condition management system 1. The monitoring assistance device 100 may be included in the terminal device 20 or the management server 200, for example.

Both the terminal device 20 and the monitoring assistance device 100 are installed, for example, in the home of the managed person. Data may be transmitted/received between the terminal device 20 and the monitoring assistance device 100 via short-range wireless communication such as NFC, Bluetooth (registered trademark), DSRC, and infrared communication.

The monitoring assistance device 100 includes, for example, a communication interface 110, an input interface 120, processing circuitry 130, and a memory 140. The communication interface 110 communicates with an external device such as the management server 200 via the communication network NW. The communication interface 110 includes, for example, a communication interface such as a network interface card (NIC).

The input interface 120 receives various input operations of an operator and outputs an electrical signal indicating the content of the received input operations to the processing circuitry 130. The input interface 120 is realized by, for example, a mouse, a keyboard, a touch panel, a track ball, a switch, buttons, a joystick, a camera, an infrared sensor, a microphone, or the like.

The input interface in the present description is not limited to those equipped with physical operation parts such as a mouse and a keyboard. For example, examples of the input interface also include an electrical signal processing circuit that receives an electrical signal corresponding to an input operation from an external input apparatus provided separately from the device and outputs the electrical signal to the control circuitry.

The processing circuitry 130 has a processor such as a central processing unit (CPU), for example. The processing circuitry 130 controls the overall operation of the monitoring assistance device 100. The processing circuitry 130 includes, for example, an acquisition function 131, a determination function 132, a notification control function 133, an update function 134, and an output function 135. The processing circuitry 130 realizes these functions by, for example, a hardware processor executing a program stored in a storage device (storage circuit).

The hardware processor is circuitry such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)), for example. Instead of storing the program in the storage device, the program may be directly embedded in the circuit of the hardware processor. In this case, the hardware processor realizes the functions by reading and executing the program embedded in the circuit. The hardware processor is not limited to being configured as a single circuit, and a plurality of independent circuits may be combined to be configured as a single hardware processor to realize each function. The storage device may be a non-transitory (hardware) storage medium. Further, a plurality of components may be integrated into a single hardware processor to realize each function.

Each component of the processing circuitry 130 may be distributed and realized by a plurality of types of hardware. The processing circuitry 130 may be realized by a processing device that can communicate with the monitoring assistance device 100 instead of a component included in the monitoring assistance device 100. Each function included in the processing circuitry 130 may be distributed to a plurality of circuits, or may be made available by starting application software stored in the memory 140.

The memory 140 is included in the storage device. The memory 140 stores workflow data 141 and execution result data 142. The workflow data 141 includes a workflow (hereinafter referred to as a device workflow) in which operation timing of the measurement device 10 is defined by the managed person. The operation timing defined in the device workflow includes an operation execution time and an operation time for each measurement device 10.

As specific operation execution time, for example, a measurement time of the sphygmomanometer 11, a measurement time of the weight and body composition meter 12, a time when the first activity meter 13 is worn, a time when the first activity meter 13 is removed, a charging start time and a charging end time of the second activity meter 14, and the like are conceivable. As the execution time, for example, the charging time of the second activity meter 14 is conceivable.

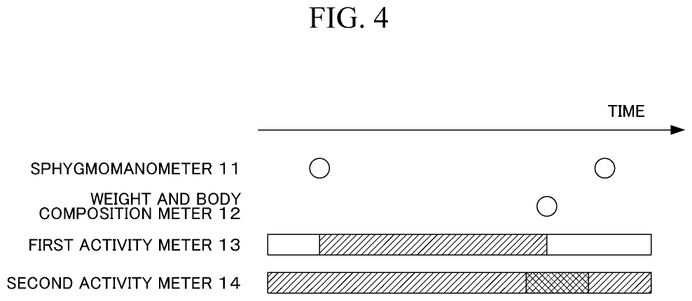

FIG. 4 is a diagram showing an example of a device workflow. In this example, a blood pressure measurement time by the sphygmomanometer 11 is set twice, 6:00 (6:00 am) and 22:00 (10:00 pm). In addition, the measurement time of the weight and body composition by the weight and body composition meter 12 is set to 20:00. Further, the wearing time (operation execution time) of the first activity meter 13 is set to 6:00, and the removal time is set to 20:00. In addition, a charging start time of the second activity meter 14 is set to 20:00, the charging end time is set to 22:00, and the charging time is set to 2 hours.

The execution result data 142 includes operation completion information of the measurement device 10. The type of the measurement device 10 is determined by information on an identifier included in the operation completion information. Specific operation completion information for each measurement device 10 includes, for example, information such as the measurement time by the sphygmomanometer 11, the measurement time by the weight and body composition meter 12, the wearing time and removal time of the first activity meter 13, and the charging start time and charging end time of the second activity meter 14.

The acquisition function 131 acquires operation execution statuses of the plurality of (plurality of types of) measurement devices 10 by receiving measurement information and operation completion information of biometric information transmitted by the terminal device 20 using the communication interface 110. The measurement information may be transmitted by the management server 200. The acquisition function 131 stores the acquired execution statuses in the memory 140 in addition to the execution result data 142. The acquisition function 131 separately acquires, for example, execution statuses of use-related operations and execution statuses of non-use-related operations of the measurement device 10. The acquisition function 131 is an example of an acquisition unit.

The determination function 132 compares a device workflow included in the workflow data 141 stored in the memory 140 with execution statuses included in the execution result data 142. The determination function 132 determines whether or not an operation of a measurement device 10 designated by a managed person in the device workflow has been performed by comparing the device workflow with the execution statuses. The determination function 132 is an example of a determination unit.

The determination function 132 determines whether or not the blood pressure or the weight and body composition of the managed person has been measured at a measurement time when the measurement device 10 is the sphygmomanometer 11 or the weight and body composition meter 12. When the measurement device 10 is the first activity meter 13, the determination function 132 determines whether the managed person has worn the first activity meter 13 at a wearing time and whether the managed person has removed the first activity meter 13 at a removal time. When the measurement device 10 is the second activity meter 14, the determination function 132 determines whether charging of the second activity meter 14 has started at a charging start time and whether charging of the second activity meter 14 has ended at a charging end time.

The notification control function 133 transmits control information to the notification device 30 and controls the notification device 30 based on whether or not an operation of the measurement device 10 has been performed. The notification control function 133, for example, controls display of the first monitor 41 to the third monitor 43 in the notification device 30, controls the sound output by the speaker 32, and controls lighting and blinking of the lamp 33. The notification control function 133 is an example of a notification control unit. The notification control function 133 includes a display control function 133A that controls display of the monitor 40. The display control function 133A is an example of a display control unit.

The notification control function 133 causes the first monitor 41 to the third monitor 43 in the notification device 30 to output a notification (for example, display) including operation-related information of each of the plurality (the plurality types) of measurement devices 10. For example, when the determination function 132 determines that an operation has been executed at an operation execution time designated by the managed person in a device workflow has been performed, the notification control function 133 switches between displays of the first monitor 41 to the third monitor 43. Display of the first monitor 41 to the third monitor 43 will be further described later.

The notification control function 133 acquires an operation execution time in the measurement device 10 based on the workflow data 141. When operation completion information, measurement information, and the like transmitted from the measurement device 10 have not yet arrived and the operation of the measurement device 10 has not been performed at the time when a scheduled execution time has elapsed from the operation execution time, the notification control function 133 switches display of any one of the first monitor 41 to the third monitor 43, which corresponds to the measurement device 10 for which the scheduled execution time has elapsed, to the execution promotion state. The scheduled execution time can be appropriately set. The scheduled execution time may be, for example, 10 minutes or 30 minutes. The notification control function 133 may control the speaker 32 and the lamp 33 of the notification device 30.

The update function 134 updates the device workflow included in the workflow data 141 when the operation completion information, the measurement information, and the like transmitted from the measurement device 10 have not yet arrived and the operation of the measurement device 10 has not been performed at the time when a postponement time has additionally elapsed after the scheduled execution time has elapsed. The postponement time can be appropriately set. The postponement time may be, for example, 30 minutes or 1 hour. The postponement time may be shorter than, longer than, or the same as the scheduled execution time.

When blood pressure measurement by the sphygmomanometer 11 included in the device workflow has not been performed, for example, the update function 134 updates the device workflow such that a process of compensating for the blood pressure measurement that has not been performed is added. The update function 134 is an example of an update unit. The update function 134 may update the workflow when the measurement device 10 is added, removed, and the like.

The update function 134 stores the updated device workflow as workflow data 141 in the memory 140. For example, when update information corresponding to an operation of the operator is output through the input interface 120 or when measurement information transmitted by the management server 200 is received, the update function 134 may update the device workflow according to the update information.

When the notification control function 133 notifies the output function 135 of operation-related information, the output function 135 transmits the notified operation-related information to the management server 200. By receiving the operation-related information, the management server 200 can check the execution status of the operation of the measurement device 10 by the managed person. The output function 135 is an example of an output unit.

The output function 135 transmits and outputs the device workflow updated by the update function 134 to the management server 200 using the communication interface 110. A medical facility in which the management server 200 to which the device workflow has been transmitted is provided can check change in the device workflow. The output function 135 may selectively output necessary output content to the management server 200 from among the operation-related information and the device workflow. The output function 135 may output all of the operation-related information and the device workflow and may receive necessary output content filtered by the management server 200.

The management server 200 is installed, for example, in a medical facility where doctors and others stay. The management server 200 receives and stores measurement information transmitted from the terminal device 20. The measurement information stored in the management server 200 can be viewed by, for example, doctors in the medical facility and is used for health management of the managed person.

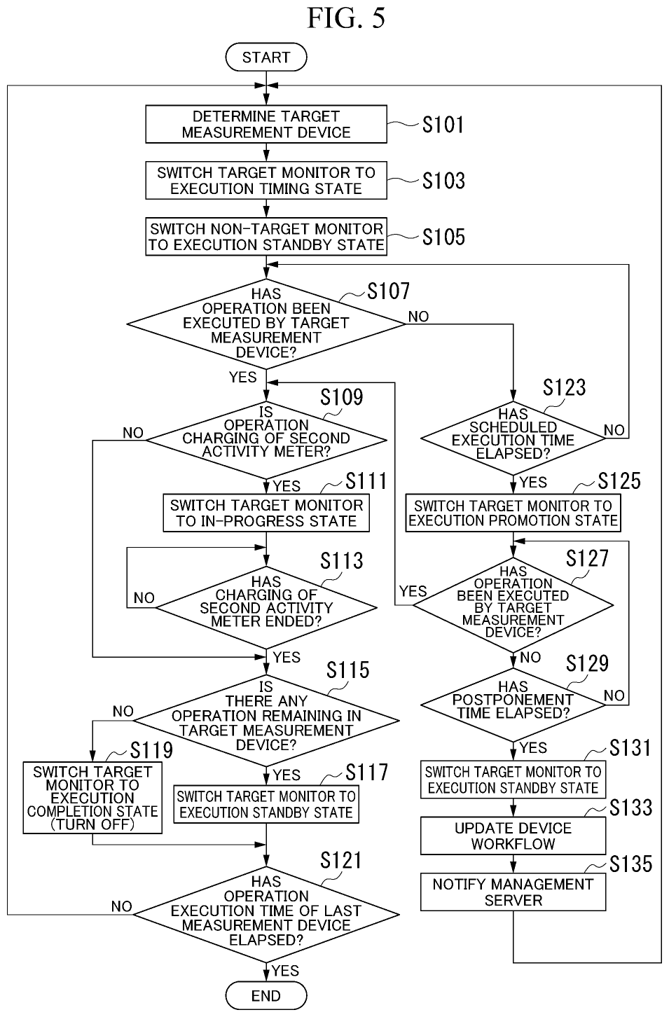

Next, processing of the monitoring assistance device 100 will be described. FIG. 5 is a flowchart showing an example of processing of the monitoring assistance device 100. The flowchart is executed once a day, for example, starting at a specific time, for example, 6:00. The flowchart may be executed once or more a day or may be executed once every few days, and the number of times of execution is appropriately defined according to the content of a device workflow, for example.

Although the content of display of the monitor 40 representing that the monitoring assistance device 100 switches the content of display of the monitor 40 corresponding to the measurement devices 10 in the order of operation execution times of the measurement devices 10 changes over time, first, the first monitor 41 to the third monitor 43 are all in the operation completion state (turned off) here.

The monitoring assistance device 100 performs interrupt processing by the acquisition function 131 during execution of the flowchart, and when measurement information and operation completion information are transmitted to the terminal device 20 and the management server 200, acquires the transmitted information. The interrupt processing may be executed at any time interval, for example, every several minutes or several tens of minutes.

When the flowchart starts, the monitoring assistance device 100 determines a measurement device 10 (hereinafter referred to as a target measurement device 10) with the closest operation execution time with reference to the device workflow in the notification control function 133 (step S101). Subsequently, when the operation execution time of the target measurement device 10 has been reached, display of the monitor 40 (hereinafter referred to as a target monitor 40) corresponding to the target measurement device 10 is switched to the execution timing state (step S103).

For example, when the target measurement device 10 is the sphygmomanometer 11, the operation execution time is, for example, 8:00. Therefore, the notification control function 133 switches display of the first monitor 41 corresponding to the sphygmomanometer 11 to the execution timing state at 8:00. Further, the notification control function 133 sets display of a monitor 40 that has not yet become a target monitor 40 (hereinafter referred to as a non-target monitor 40) to the execution standby state (step S105).

Subsequently, the determination function 132 compares the measurement information and the operation completion information acquired by the acquisition function 131 with the device workflow stored in a storage 109 to determine whether or not the operation of the target measurement device 10 has been performed (step S107). For example, when the target measurement device 10 is the sphygmomanometer 11, the notification control function 133 determines whether or not the blood pressure of a subject P has been measured by the sphygmomanometer 11.

When the determination function 132 determines that the operation of the target measurement device 10 has been executed, the notification control function 133 determines whether or not the executed operation is charging of the second activity meter 14 based on the measurement information and the operation completion information acquired by the acquisition function 131 (step S109). When it is determined that the executed operation is not charging of the second activity meter 14, for example, when it is determined that the executed operation is blood pressure measurement by the sphygmomanometer 11, processing of the notification control function 133 proceeds to step S115.

When it is determined that the executed operation is charging of the second activity meter 14, display of the third monitor 43 corresponding to the second activity meter 14 becomes the execution timing state, and the notification control function 133 switches the display of the third monitor 43 to the in-progress state (step S111). The notification control function 133 switches the display of the third monitor 43 to the in-progress state, and at the same time outputs in-progress information to the management server 200.

Subsequently, the determination function 132 determines whether or not charging of the second activity meter 14 has ended based on the measurement information and the operation completion information acquired by the acquisition function 131 (step S113). When it is determined that charging of the second activity meter 14 has not ended, the determination function 132 repeats processing of step S113.

When it is determined that charging of the second activity meter 14 has ended, the determination function 132 determines whether or not there is any operation remaining in the target measurement device 10 with reference to the device workflow stored in the storage 109 (step S115). For example, if the target measurement device 10 is the sphygmomanometer 11 and measurement to be performed at 8:00 has ended, measurement to be performed at 22:00 remains, and thus the determination function 132 determines that there is an additional operation remaining in the target measurement device 10.

When the determination function 132 determines that there is an operation remaining in the target measurement device 10, the notification control function 133 switches display of the target monitor 40 to the execution standby state (step S117). When the determination function 132 determines that there is no operation remaining in the target measurement device 10, the notification control function 133 turns off the target monitor 40 to set it to the operation completion state (step S119).

Subsequently, the determination function 132 determines whether or not the operation execution time of the last measurement device 10 has elapsed with reference to the device workflow stored in the storage 109 (step S121). Upon determining that the operation execution time of the last measurement device 10 has not elapsed, the determination function 132 returns processing to step S101. When the determination function 132 determines that the operation execution time of the last measurement device 10 has elapsed, the monitoring assistance device 100 ends the processing shown in FIG. 5.

When it is determined in step S107 that the operation of the target measurement device 10 has not been performed, the determination function 132 determines whether or not the scheduled execution time has elapsed (step S123). Upon determining that the scheduled execution time has not elapsed, the determination function 132 returns processing to step S107. Subsequently, the determination function 132 repeatedly executes processing of steps S107 and S123.

When the determination function 132 repeatedly executes processing of steps S107 and S123 and determines that the scheduled execution time has elapsed, the notification control function 133 switches display of the target monitor 40 to the execution promotion state (step S125). Subsequently, the determination function 132 compares the measurement information and the operation completion information acquired by the acquisition function 131 with the device workflow stored in the storage 109 to determine whether or not the operation of the target measurement device 10 has been performed (step S127).

Upon determining that the operation of the target measurement device 10 has been performed, processing of the determination function 132 proceeds to step S109. Upon determining that the operation of the target measurement device 10 has not been performed, the determination function 132 determines whether or not the postponement time has passed (step S129). Upon determining that the postponement time has not elapsed, the determination function 132 returns processing to step S127. Subsequently, the determination function 132 repeatedly executes processing of steps S127 and S129.

When the determination function 132 determines that the postponement time has elapsed, the notification control function 133 switches display of the target monitor 40 to the execution standby state (step S131). Subsequently, the update function 134 updates the workflow set in the target measurement device 10 depending on the content of an operation that has not been executed (step S133).

The update function 134 updates the workflow such that, for example, the operation that has not been executed is scheduled to be executed after a predetermined time, for example, after 6 hours. The workflow may be updated in any manner. For example, device workflows may be updated such that operations that have not been executed are newly scheduled to be executed uniformly after a few hours. Alternatively, an operation execution time may be newly designated for each measurement device 10, and a device workflow may be updated such that the designated time is set as the operation execution time.

After the update function 134 updates the device workflow, the output function 135 outputs the updated device workflow by transmitting it to the management server 200 (step S135). Subsequently, the update function 134 returns processing to step S101. Thereafter, the monitoring assistance device 100 ends the processing shown in FIG. 5 by executing processing up to step S121.

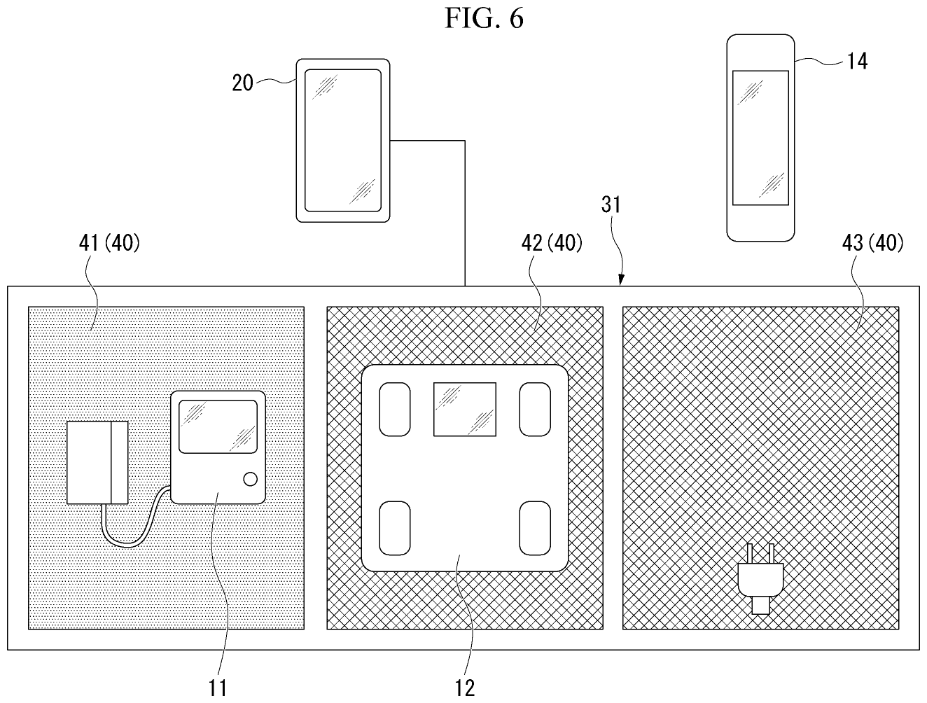

Next, transition of content displayed on the monitor 40 of the guide sheet 31 while processing by the monitoring assistance device 100 is being performed will be described. FIG. 6 to FIG. 10 are diagrams showing display content of the monitor 40. Transition of the display content of the monitor 40 occurs, for example, in the order shown in FIG. 6 to FIG. 10 over time.

For example, when all operations performed by the measurement devices 10 on the previous day have been executed, the first monitor 41 to the third monitor 43 are all turned off and enter the execution completion state. In this state, if the operation execution time of the sphygmomanometer 11, which is the measurement device 10 with the earliest operation execution time, has come, for example, the operation execution timing state is displayed on the first monitor 41 corresponding to the sphygmomanometer 11, as shown in FIG. 6, and display of the first monitor 41 is switched from the execution completion state in which it is turned off to the execution timing state. At this time, display of the second monitor 42 corresponding to the weight and body composition meter 12 and display of the third monitor 43 corresponding to the second activity meter 14 are switched from the execution completion state in which they are turned off and to the execution standby state.

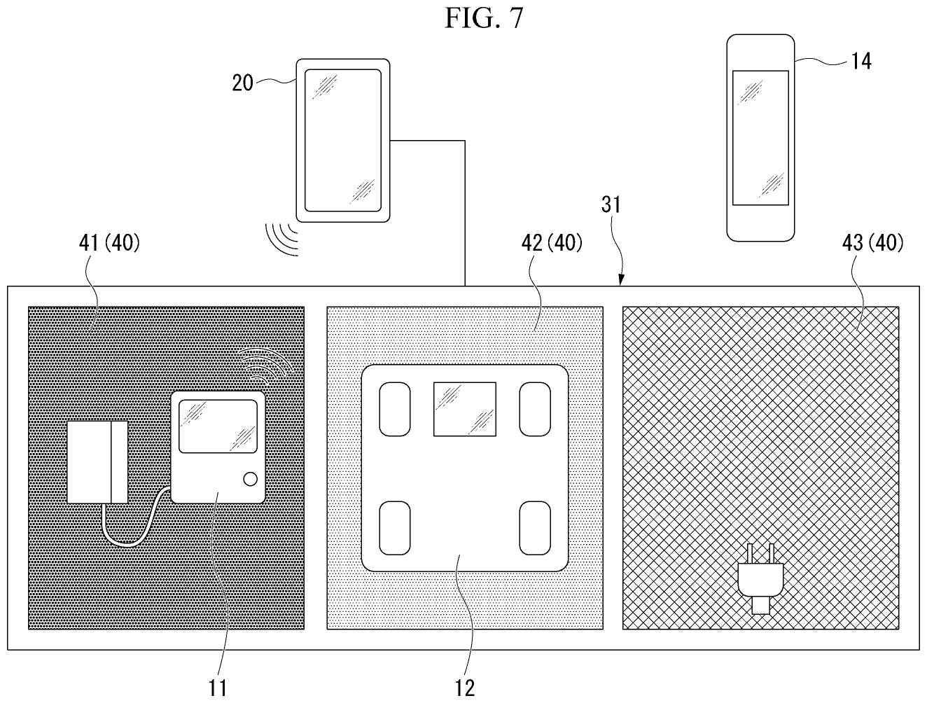

Subsequently, it is assumed that a managed person performs blood pressure measurement using the sphygmomanometer 11 until the operation execution time of the sphygmomanometer 11, and the acquisition function 131 acquires operation completion information of the sphygmomanometer 11 transmitted from the terminal device 20. In this case, the first monitor 41 is turned off and enters the execution completion state, as shown in FIG. 7. Thereafter, if the measurement device 10 having the next earliest operation execution time is the weight and body composition meter 12, display of the second monitor 42 is switched from the execution standby state to the execution timing state. Display of the third monitor 43 corresponding to the second activity meter 14 remains in the execution standby state.

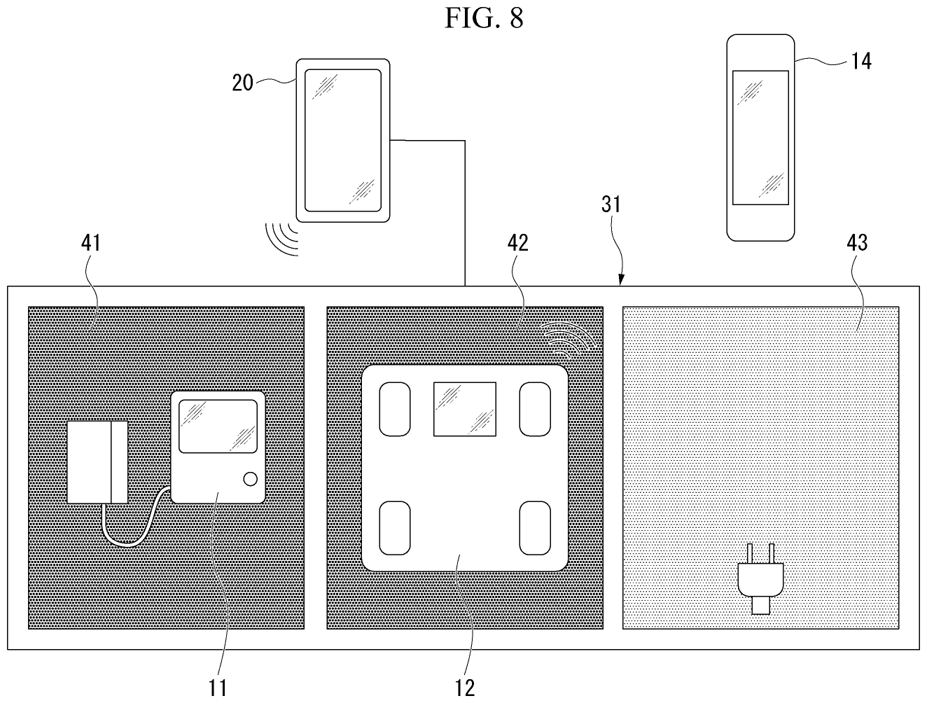

Subsequently, it is assumed that the managed person performs measurement of the weight and body composition using the weight and body composition meter 12 until the operation execution time of the weight and body composition meter 12, and the acquisition function 131 acquires operation completion information of the weight and body composition meter 12 transmitted by the terminal device 20. In this case, the second monitor 42 is turned off and enters the execution completion state, as shown in FIG. 8. Thereafter, if the measurement device 10 having the next earliest operation execution time is the second activity meter 14, display of the third monitor 43 is switched from the execution standby state to the execution timing state. The first monitor 41 remains in the turned off state.

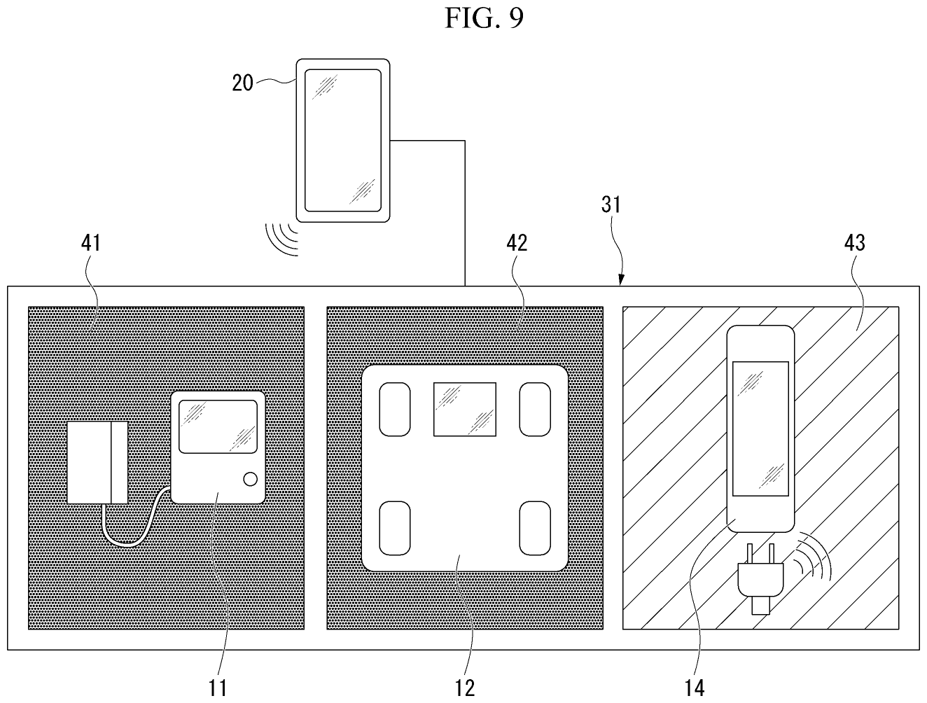

Subsequently, it is assumed that the managed person connects the second activity meter 14 to the charging device and starts charging until the operation execution time of the second activity meter 14, and the acquisition function 131 acquires operation completion information of the second activity meter 14 transmitted by the terminal device 20. In this case, display of the third monitor 43 is switched from the execution timing state to the in-progress state, as shown in FIG. 9. Display of the first monitor 41 and display of the second monitor 42 remain in the turned off state.

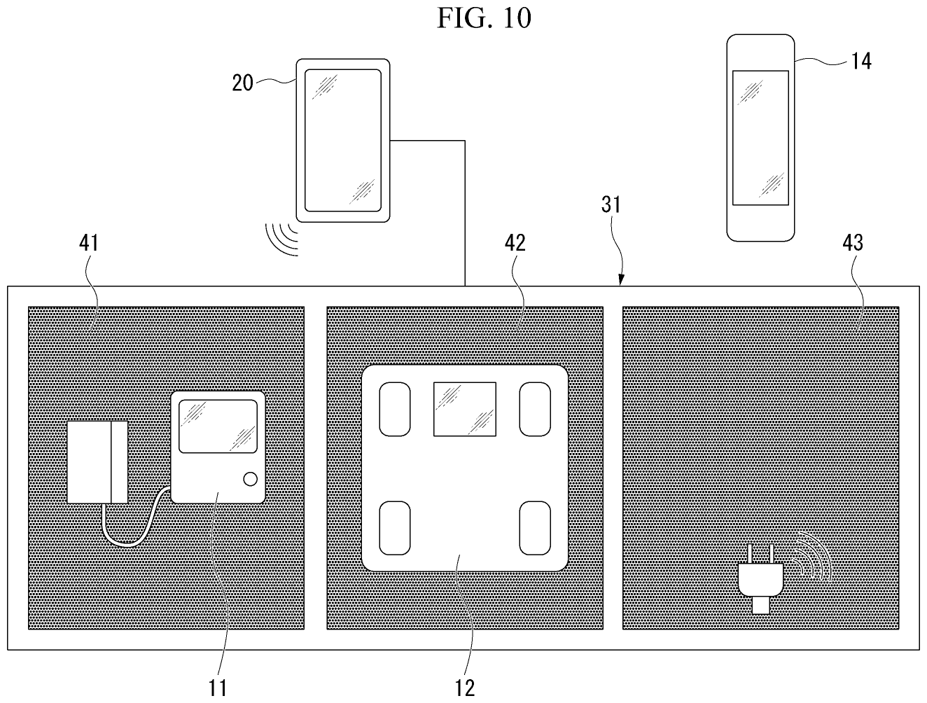

Subsequently, it is assumed that the managed person removes the second activity meter 14 from the charging device and ends charging until operation execution time (charging end time) of the second activity meter 14, and the acquisition function 131 acquires operation completion information of the second activity meter 14 transmitted by the terminal device 20. In this case, the third monitor 43 is turned off and enters the execution completion state, as shown in FIG. 10. In this way, all of the first monitor 41 to the third monitor 43 are turned off to indicate the execution completion state.

The monitoring assistance device 100 according to the embodiment controls the notification device 30 and outputs a notification including operation-related information on the basis of a device workflow that defines execution statuses of operations of the measurement devices 10 and operation timing of the measurement devices 10. For example, when the managed person measures the blood pressure using the sphygmomanometer 11 at the time when the operation execution time (measurement time) of the sphygmomanometer 11 has come, operation completion notification is performed.

If the managed person does not measure the blood pressure using the sphygmomanometer 11 at the operation execution time, the monitoring assistance device 100 causes the notification device 30 to display the execution promotion state through the notification control function 133. Therefore, it is possible to prompt the managed person to perform an operations of a measurement devices 10, and since execution timing statuses are sequentially displayed for measurement devices 10 that need to execute operations, it is difficult for the managed person to misrecognize the measurement order of the measurement devices 10.

Although the notification device 30 performs notification on the basis of the execution statuses of the operations of the measurement devices 10 at the time when an operation execution time has come in the above-described embodiment, the notification device 30 may perform notification before the operation execution time. For example, the notification device 30 may be caused to output a notification including the operation execution time a certain period of time, for example, 10 minutes before the operation execution time. In this case, the managed person can ascertain that the operation of the measurement device is necessary before the operation execution time, and thus the managed person can certainly be aware of that it is time to execute the operation of the measurement device 10.

Although the measurement device 10 is placed on the corresponding monitor 40 in the above-described embodiment, the measurement device 10 may not be placed on the monitor 40 and the monitor 40 may display a notification. Although the notification is displayed in a color on the monitor 40, the notification may be made according to a display form other than color. For example, the notification may be a text message, or may be a photograph or an illustration image showing the measurement device 10.

Although the monitor 40 includes a plurality of monitors, the first monitors 41 to the third monitors 43, corresponding to the measurement devices 10 in the above- described embodiment, there may be one monitor 40. In this case, the monitor 40 may display the features, for example, the names, exterior shapes, and the like of the measurement devices 10 that are operation targets.

Although the monitor 40 is provided on the guide sheet 31 in the above- described embodiment, the monitor 40 may be provided on a place other than the guide sheet 31, and may be provided on a display included in the terminal device 20 or the monitoring assistance device 100, for example. Although the first monitor 41 to the third monitor 43 in the monitor 40 are adjacently provided, the first monitor 41 to the third monitor 43 may be provided apart from each other. The first monitor 41 to the third monitor 43 may be independently provided in different places.

Although notification is made when an operation has been executed, an operation has not been executed, or the like at the time when an operation execution time has come according to a device workflow in the above-described embodiment, notification timing may be determined in another aspect. The notification timing may be determined, for example, by the order of operation execution times of the measurement devices 10, or may be determined by the operation execution times and the order thereof. Further, the notification timing may be set including results of the operations executed by the measurement devices 10. Further, the notification timing may be set including instructions from a doctor through the management server 200, and the like.

Although the display content of the monitor 40 includes display of the execution completion state, execution timing state, execution standby state, in-progress state, and execution promotion state in the above-described embodiment, the display content may include notification other than these, for example, information on operation such as a failure (communication error). Further, the monitor 40, the speaker, and the lamp in the notification device 30 may be installed apart from each other in a distributed manner.

Although the notification control function 133 outputs a notification including operation-related information of the measurement devices 10 in the order in which operation execution time indicated in the device workflow is closest, timing of notification of operation-related information may be determined based on criteria other than the order in which the operation execution time is closest. For example, the timing of notification of operation-related information may be determined including measurement results of the measurement devices 10.

Alternatively, the timing to notification of operation-related information may be determined including the support of an administrator (doctor or the like), or the timing of notification of operation-related information may be determined by combining these. In addition, notification of operation-related information may include information on operation such as uncharged, charged, and failure (communication error) in addition to execution standby, execution timing, and the like.

Although the notification device 30 is provided independently of the terminal device 20 and the monitoring assistance device 100 in the above-described embodiment, a part or all of the notification device 30 may be provided in the terminal device 20 or the monitoring assistance device 100. Further, the mode of notification by the notification control function 133 may be changed on the basis of measurement information.

For example, for a managed person with a high blood pressure, a strong notification (notification of brightening the brightness of display or blinking faster) may be performed. Alternatively, display of the execution promotion state in the case where the operations of the measurement devices 10 are not performed may be more emphasized than other operation-related information (brightness is improved or sound is increased), or a blinking interval at the time of displaying the execution promotion state may be shortened. In addition, although the charging start time is fixed, the remaining charge amount in the second activity meter 14 may be estimated according to the amount of activity of the managed person, and the like, and the charging start time may be determined on the basis of the estimated remaining charge amount.

According to at least one embodiment described above, it is possible to prompt a managed person to perform operations of measurement devices by including an acquisition unit configured to acquire an execution status of at least one of an operation related to use of an apparatus that acquires biometric information of the managed person or an operation other than the operation related to use, a determination unit configured to determine, on the basis of the execution status of the managed person and a workflow defining operation timing of the apparatus, whether or not the operation has been executed at the operation timing, and a notification control unit configured to control a notification device that outputs a notification including information on the operation of the apparatus on the basis of whether or not the operation has been executed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A monitoring assistance device comprising processing circuitry configured to:
acquire an execution status of at least one of an operation related to use of an apparatus that acquires biometric information of a managed person or an operation other than the operation related to use;
determine, on the basis of the execution status of the managed person and a workflow defining an operation timing of the apparatus, whether or not the operation has been executed at the operation timing; and
control a notification device that outputs a notification including information on the operation of the apparatus on the basis of whether or not the operation has been executed.

2. The monitoring assistance device according to claim 1, wherein the processing circuitry is configured to acquire execution statuses of operations of a plurality of apparatuses and cause the notification device to output a notification including information on the operation of each of the plurality of apparatuses.

3. The monitoring assistance device according to claim 1, wherein the notification device comprises a placing part on which the apparatus is placed, and an information display provided on the placing part,
wherein the processing circuitry is configured to cause the information display to display information on the operation other than the operation related to use of the apparatus.

4. The monitoring assistance device according to claim 1, wherein the apparatus comprises a measurement apparatus that measures a living body,
wherein the processing circuitry is configured to acquire a measurement status of the biometric information of the managed person by the apparatus as the execution status of the operation related to use of the apparatus, and determine whether or not the biometric information of the managed person has been measured by the measurement apparatus.

5. The monitoring assistance device according to claim 1, wherein the apparatus comprises a chargeable apparatus having a charging function,
wherein the processing circuitry is configured to acquire a charging start status of the chargeable apparatus as the execution status of the operation other than the operation related to use of the apparatus, and determine whether or not charging of the chargeable apparatus has started.

6. The monitoring assistance device according to claim 1, wherein the notification device comprises a display device that displays information as the notification, and
the processing circuitry is configured to cause the display device to display information on the operation of the apparatus.

7. The monitoring assistance device according to claim 6, wherein the processing circuitry is configured to cause the display device to display different information depending on the execution status of the operation as notification of the information on the operation of the apparatus.

8. The monitoring assistance device according to claim 1, wherein the processing circuitry is configured to cause the notification device to output reminder information when it is determined that the operation of the apparatus has not been executed.

9. The monitoring assistance device according to claim 1, wherein the processing circuitry is configured to update the workflow when it is determined that the operation of the apparatus has not been executed.

10. The monitoring assistance device according to claim 9, wherein the processing circuitry is configured to output the updated workflow to a management server that manages the managed person.

11. A monitoring assistance system comprising:
processing circuitry; and
a notification device,
wherein the processing circuitry is configured to:
acquire an execution status of at least one of an operation related to use of an apparatus that acquires biometric information of a managed person or an operation other than the operation related to use;
determine, on the basis of the execution status of the managed person and a workflow defining an operation timing of the apparatus, whether or not the operation has been executed at the operation timing; and
control the notification device, and
the notification device is configured to output a notification including information on the operation of the apparatus on the basis of whether or not the operation has been executed.

12. A monitoring assistance method, using a computer, comprising:
acquiring an execution status of at least one of an operation related to use of an apparatus that acquires biometric information of a managed person or an operation other than the operation related to use;
determining, on the basis of the execution status of the managed person and a workflow defining an operation timing of the apparatus, whether or not the operation has been executed at the operation timing; and
controlling a notification device that outputs a notification including information on the operation of the apparatus on the basis of whether or not the operation has been executed.

13. A computer-readable non-transitory storage medium storing a program causing a computer to:
acquire an execution status of at least one of an operation related to use of an apparatus that acquires biometric information of a managed person or an operation other than the operation related to use;
determine, on the basis of the execution status of the managed person and a workflow defining an operation timing of the apparatus, whether or not the operation has been executed at the operation timing; and
control a notification device that outputs a notification including information on the operation of the apparatus on the basis of whether or not the operation has been executed.
